(19) United States
(12) Patent Application Publication
(10) Pub. No.: US 2025/0228583 A1
(43) Pub. Date: Jul. 17, 2025

(54) CLOT AND FOREIGN BODY REMOVAL SYSTEM AND METHODS FOR USE

(71) Applicant: Yince Loh, Seattle, WA (US)

(72) Inventor: Yince Loh, Seattle, WA (US)

(21) Appl. No.: 19/059,198

(22) Filed: Feb. 20, 2025

Related U.S. Application Data
(63) Continuation-in-part of application No. 18/045,056, filed on Oct. 7, 2022, now abandoned.
Application No. 18/045,056 is a continuation of application No. 14/837,787, filed on Aug. 27, 2015, now abandoned.
(60) Provisional application No. 62/056,359, filed on Sep. 26, 2014.
(60) Provisional application No. 62/068,858, filed on Oct. 27, 2014.

(51) Int. Cl. A61B 17/221; A61B 17/00; A61B 17/22; A61B 17/30

(52) U.S. Cl. CPC A61B 17/221; A61B 2017/00867; A61B 2017/22079; A61B 2017/2212; A61B 2017/306; A61F 2210/0014; A61F 2230/0091

(57) ABSTRACT

A highly versatile removal system for thrombus and foreign body removal, which is capable of many possible configurations, some of which are a partially constrained configuration; 2) a clot trapping, or “en bloc”, configuration or 3) an enmeshed configuration.

### PRIORITY DATA

This application claims priority from the following U.S. patent applications: this application is a continuation-in-part application of U.S. Ser. No. 18/045,056 filed on Oct. 7, 2022; which is a continuation of U.S. application Ser. No. 14/837,787, filed Aug. 27, 2015; which claims the benefit of U.S. Provisional Application Ser. No. 62/056,359, filed on Sep. 26, 2014, and U.S. Provisional Application Ser. No. 62/068,858, filed on Oct. 27, 2014. The disclosures of each application are incorporated herein, by reference, in their entirety.

### FIELD OF INVENTION

This invention pertains generally to acute stroke intervention, and more particularly to systems, devices and methods for thrombus or foreign body removal.

### BACKGROUND

The U.S. is taking an exponentially increasing interest in acute stroke interventions evidenced by its Medicare reimbursement rates, and push for primary and comprehensive stroke center designations. Acute stroke interventions may have significant cost effectiveness and benefit quality of life. U.S. healthcare dollars that are spent on acute interventions are a paltry sum when compared to the lifelong long-term care needs of the victim of a massive, disabling stroke. Medical device companies are aware of this new trend in Medicare reimbursement and are making stroke intervention their new priority. There is a need for a next generation acute stroke intervention device that can be used to treat patients quickly and effectively.

There have been many attempts to develop and treat acute strokes such as improving the blood circulation distal to a cerebral embolus (the clot that causes the stroke). This first started with intravenous infusion of fibrinolysis agents. Physicians have also described ultrasonography or endovascular techniques (from within the blood vessel through catheters and wires inserted in the groin) to include infusion of rt-PA or clot maceration with micro-snares or balloons.

A few systems have been designed exclusively for removal of the above-described clot. The Merci Removal System® was designed and conceived at UCLA by Dr. Y. Pierre Gobin, and manufactured by Concentric Medical, Inc, (now Stryker Medical); this product has been discontinued. The Merci Removal System® is comprised of three products: the Merci Retriever®, Merci® Balloon Guide Catheter, and Merci® Microcatheter. The three products are used together in a procedure with the goal of removing a foreign body from an affected vessel. Generally, physicians have used the Merci Removal System® to treat clots in brain arteries. Once the location of a clot has been identified using angiography, the Merci® Balloon Guide Catheter is inserted, by way of a small incision in the groin, into the femoral artery. Under x-ray guidance, the Merci® Balloon Guide Catheter is maneuvered up to the carotid artery in the neck, a micro-guidewire and the Merci® Microcatheter are deployed through the Merci® Balloon Guide Catheter and placed just beyond the clot. The Merci® Retriever device is deployed to engage and ensnare the clot. Once the clot is captured, the Merci® Balloon Guide Catheter is inflated to temporarily arrest forward flow while the clot is being withdrawn. The clot is pulled into the Merci® Balloon Guide Catheter and removed from the patient's body. The balloon is deflated, and blood flow is restored. The Merci Removal System® can also be used to retrieve foreign bodies in the peripheral, coronary, and neuro vasculature.

The Merci Removal System® has been studied at UCLA and shown safety and efficacy both with and without additional use of intravenous rat-PA. Unfortunately, however, only about half the patients symptomatic from a vessel occlusion can be effectively recanalized with the Merci Removal System®. Clinical improvements are marked in the recanalized group.

The second generation of intracranial thrombectomy device is the aspiration catheter (the “Max” line), manufactured by Penumbra, Inc. Once the location of the clot has been identified using angiography, a larger guiding catheter is inserted, by way of a small incision in the groin, into the femoral artery. Under x-ray guidance, this catheter is maneuvered up to the carotid artery in the neck. An introducing micro-guidewire and a microcatheter are deployed through the aspiration catheter, which are together advanced into the guiding catheter then placed just up against the clot. The physician then removes the introducing micro-guidewire and microcatheter and attaches the aspiration catheter to a suction assembly to aspirate the clot. A separator wire can be used to assist in the debulking of the aspiration catheter tip if it is completely occluded by clot. Alternatively, the aspiration catheter can be removed en bloc with the clot if its tip is occluded. The Penumbra system has been shown to be safe and effective. Similar to the Merci data, not all patients could be recanalized with the system, but the clinical improvements were marked in the recanalized group.

The latest generations of intracranial thrombectomy devices are known as “stent-trievers”. These are non-detachable, fully recoverable stents that are attached distally to a wire. Solitaire is a stent-triever manufactured by ev3, now part of Covidien. Trevo is another stent-triever manufactured by Concentric, now Stryker Medical. The two are similar in concept and overall application and are described as one device herein. Once the location of the clot has been identified using angiography, a balloon guiding catheter is inserted, by way of a small incision in the groin, into the femoral artery. Under x-ray guidance, this catheter is maneuvered up to the carotid artery in the neck. A micro-guidewire and a microcatheter are together advanced into a guiding catheter and then advanced past the clot. The micro-guidewire is removed, and the stent-triever is advanced up to the microcatheter tip. The stent-triever is optimally positioned to engage the clot once unsheathed (pulling the catheter off of the device to let it take its native shape). In its expanded position, the stent-triever temporarily restores flow alongside the clot. Once the clot is engaged by the stent-triever struts, the balloon on the guiding catheter is inflated to temporarily arrest forward flow while the clot is being withdrawn. The clot is pulled into the balloon guiding catheter and completely out of the body. The balloon is then deflated, and blood flow is restored. Both stent-triever systems are safe and effective. However, not all patients can be recanalized with the system; clinical improvements were marked in the recanalized group. These prior systems are limited in their effectiveness because the blood vessel of the patient is often not recanalized after a first attempt, and several attempts or different methods may need to be employed to fully remove the clot. When differing methods are employed, this wastes time because the surgeon often needs to fully remove the implement he was working with and introduce a new implement, with a different way of attempting to remove or macerate the clot.

Accordingly, there is a need for a device that provides a surgeon multiple options to address a clot, such as ensnaring, macerating or separating the thromboembolism or other objects from within a patient's cerebral blood vessel, without having to withdraw and re-insert a separate implement, thereby improving the chances for recanalization of a blood vessel with limited passes.

### SUMMARY

An object of the invention is to provide a system, device and method that allows for improved mechanical extraction or maceration of clot (thrombus) or foreign body from within a brain blood vessel, either arterial or venous. Another object of the invention is to safely retrieve clots from brain arteries during an acute stroke. Another object of the invention is to provide a system that can be used in conjunction with an aspiration catheter. Another object of the invention is to provide a system that can provide flow around a clot if removal is not possible.

### DRAWINGS

Objects, features and advantages of the present invention will become apparent in the following description with reference to the accompanying drawings, of which:

FIG. 1 is a perspective view of an embodiment of a clot removal system of the present invention;

FIG. 2 is a side view of an embodiment of a clot removal system of the present invention;

FIG. 3 is a left, end view of the clot removal system depicted in FIG. 2;

FIG. 4 is a side view of an embodiment of a clot removal system of the present invention, where a clot removal device of the system is disposed and compressed within a sheath;

FIG. 5 depicts a common femoral arterial vascular pathway used to access the cerebral circulation for acute stroke thrombectomy and other cerebrovascular procedures;

FIG. 6 is a longitudinal, partial cross-sectional view schematically illustrating an intracorporeal lumen having therein, an aspiration catheter, a microcatheter and a guidewire positioned in a in relation to a clot identified for removal;

FIG. 7 is a view similar to FIG. 6, but illustrating a subsequent step where the guidewire is removed and a clot removal device of the present invention is disposed and compressed within the microcatheter;

FIG. 8A is a view similar to FIG. 7, but illustrating a subsequent step in a method for dislodgement and removal of the clot, where the clot removal device is in a partially constrained configuration;

FIG. 8B is a view similar to FIG. 8A, but illustrating a subsequent step in a method for dislodgement and removal of the clot, where the clot removal device is being moved back and forth (i.e. proximally and distally) and the clot removal device is being depicted in a proximally retracted position to macerate the clot;

FIG. 8C is a view similar to FIG. 8B, but illustrating a subsequent step in a method for dislodgement and removal of the clot, where the clot removal device is being moved back and forth (i.e. proximally and distally) and the clot removal device is being depicted in a distally advanced position to macerate the blood clot;

FIG. 8D is a view similar to FIG. 8C, but illustrating a subsequent step in a method for dislodgement and removal of the clot, where the clot removal system of the present invention has fully removed the clot from the intracorporeal lumen;

FIG. 9A is a view similar to FIG. 7, but illustrating a subsequent step in a method for dislodgement and removal of the clot, where the clot removal device is in a clot trapping configuration;

FIG. 9B is a view similar to FIG. 9A, but illustrating a subsequent step in a method for dislodgement and removal of the clot, where the clot removal device is moved back (i.e. proximally) to engage the clot and trap the clot between the clot removal device and an aspiration catheter;

FIG. 9C is a view similar to FIG. 9B, but illustrating a subsequent step in a method for dislodgement and removal of the clot, where the clot removal system of the present invention is being used to move the clot proximally to remove the clot from the body;

FIG. 10A is a view similar to FIG. 7, but illustrating a subsequent step in a method for dislodgement and removal of the clot, where the clot removal device is in a configuration where the clot enmeshes the clot removal device; and

FIG. 10B is a view similar to FIG. 10A, but illustrating a subsequent step in a method for dislodgement and removal of the clot, where the clot removal device is moved back (i.e. proximally), enmeshed with the clot, and engages an aspiration catheter.

### DETAILED DESCRIPTION OF THE INVENTION

The present invention, a clot or foreign body removal system (“removal system”) and methods for use, is described more fully hereinafter with reference to the accompanying drawings, in which preferred embodiments of the invention are shown. This invention may, however, be embodied in many different forms and should not be construed as limited to the embodiments set for herein; rather, these embodiments are provided so that this disclosure will be thorough and complete and will fully convey the scope of the invention to those skilled in the art. Although this device and methods are applicable for use in removing both clots and foreign bodies, they are referred to, generally, as clots hereinafter.

The inventive system operates on the concept of mechanical thrombectomy (physical removal of clot) and addresses hurdles of the Merci Removal System® and other prior devices. These hurdles include: (1) the manner in which a distal device engages the clot; (2) the manner in which the system interacts with the proximal brain vessel anatomy; (3) minimizing the distance of removal; (4) reducing vessel trauma when macerating clot under aspiration and (5) improving distal engagement of clot.

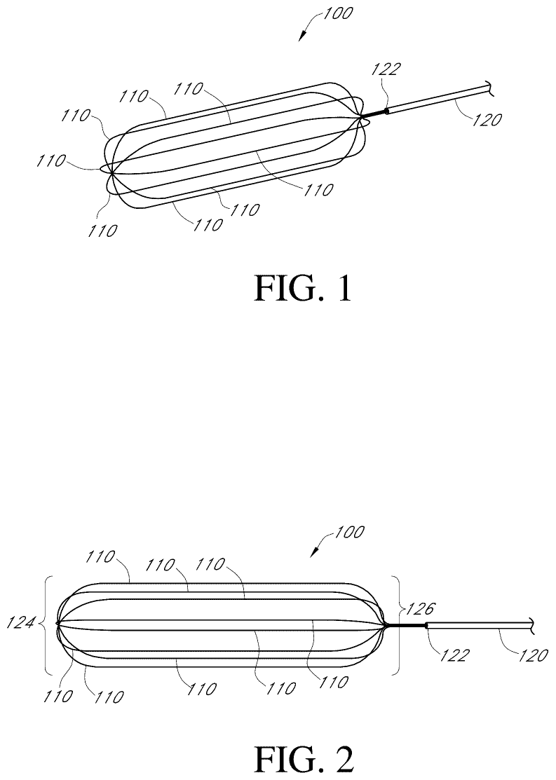

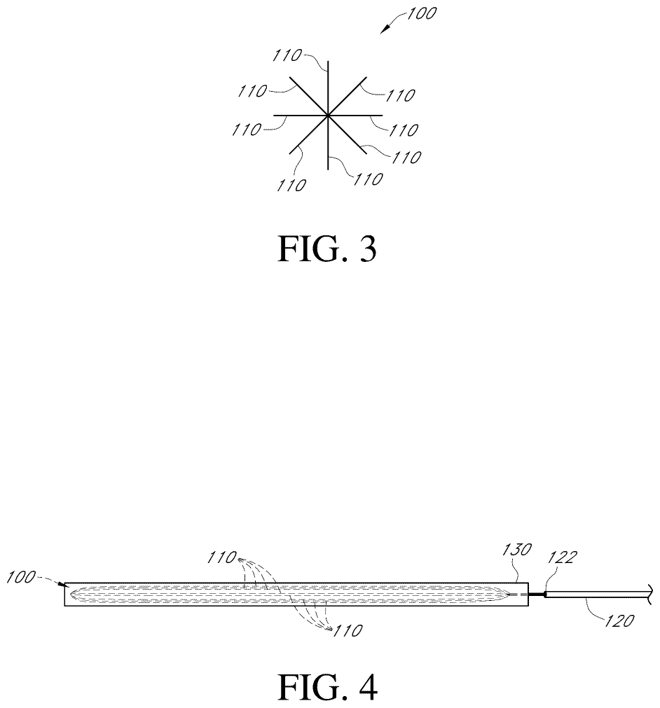

Referring to FIGS. 1-3, an embodiment of a clot removal system of the present invention is depicted. The clot removal system includes a clot removal device 100, and the clot removal device 100 of the embodiment depicted includes several legs 110 that form the whisk-like structure of the clot removal device 100. In this embodiment, the clot removal device 100 has four loops that form eight legs 110. The clot removal device 100 may have any structure or any number of legs 110, and the legs 110 can be formed as individual elements. The legs 110 do not have to be part of a loop. In this embodiment, the four loops, and thereby the eight legs 110, are connected to each other at both ends of the clot removal device 100. Further, in this embodiment, the four loops form oval rings, and the eight legs 110 of the four loops are symmetrically spaced from one another. The legs 110 may be made from any material that is medical grade that is compressible and expands, when decompressed, to form and maintain a rigid structure (e.g., a shape memory alloy material). In this embodiment, the legs 110 are made of medical grade nitinol, a nickel and titanium alloy. The clot removal device 100 is connected to a pusher wire 120. In this embodiment, preferably, the pusher wire 120 has a 0.014″ to 0.016″ diameter. The clot removal device 100, as part of the clot removal system, may be connected to the pusher wire 120 in many different ways. In the embodiment depicted, extensions of the legs 110 extend into the pusher wire 120 at a connection point 122. In another second embodiment, an extension from the whisk-like structure of the clot removal device 100 may be welded directly to the pusher wire 120 at the connection point 122. In this second embodiment, an annular ring encases the whisk-like structure extension and the pusher wire 120 at the connection point 122, and the whisk-like structure extension and the pusher wire 120 are approximately the same diameter. In one embodiment, the legs 110 can be configured to detach at the connection point 122 using a mechanical or electrolytic mechanism, such as known embolization coils, to allow permanent implantation of the clot removal system 100 if the clot 154 cannot be removed. Referring to FIG. 2, when expanded, the legs 110 on one end of the clot removal device 100 form a distal cap 124, and the legs 110 on the other end of the clot removal device 100 form a cutting cage 126.

Referring now to FIG. 4, in conjunction with FIGS. 1-3, the clot removal device 100 is moveable between a fully expanded state as illustrated in FIGS. 1-3, and a compressed state as illustrated in FIG. 4. In this embodiment, when stored and for transport, the clot removal device 100 is compressed and inserted into a sheath 130. The sheath 130 keeps the clot removal device 100 in a compressed state until ready for use (FIG. 4).

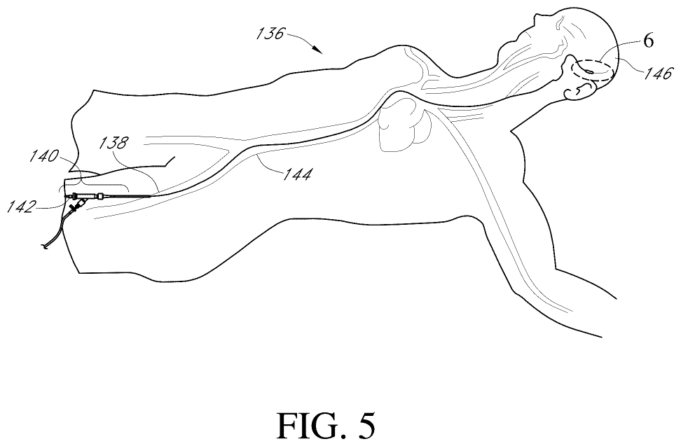

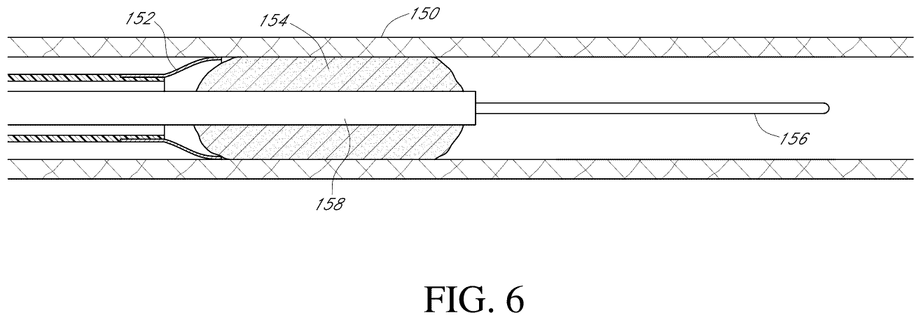

Referring now to FIGS. 5-7, the delivery of the clot removal device 100 to a clot is depicted. As is known in the art, a small incision is made in the in the groin of a body 136 of patient. The small incision creates a body entry point 138 for medical tools to be inserted into, and extracted from, a femoral artery 144 for a surgeon to access the brain 146 of a patient. As depicted in FIG. 6, once the location of a clot 154 in an intracorporeal lumen 150 in the brain 146 has been identified using angiography, in this embodiment, the surgeon, using a guidewire 156, moves an aspiration catheter 152 into contact with the clot 154. The surgeon also places a microcatheter 158 into location using the guidewire 156. The medical tools, such as the aspiration catheter 152, the guidewire 156, the microcatheter 158 and the clot removal device 100, as explained below, are inserted into and removed from the body 136 through the entry point 138. These devices are all controlled by hubs which are located at a hub assembly 140 near the entry point 138. Further, the clot removal device 100 of this embodiment includes a locking mechanism 142 in the hub assembly 140. With the microcatheter 158 in place in the clot 154 (FIG. 6), the guidewire 156 is removed. The surgeon or a medical practitioner, then, inserts the clot removal device 100 into the proximal end of the microcatheter 158 outside the body in the hub assembly 140 and removes the sheath 130. Once the sheath 130 is removed, the clot removal device 100 naturally expands a little until the clot removal device 100 touches the inner sidewalls of the microcatheter 158. The clot removal device 100 is still compressed at this point.

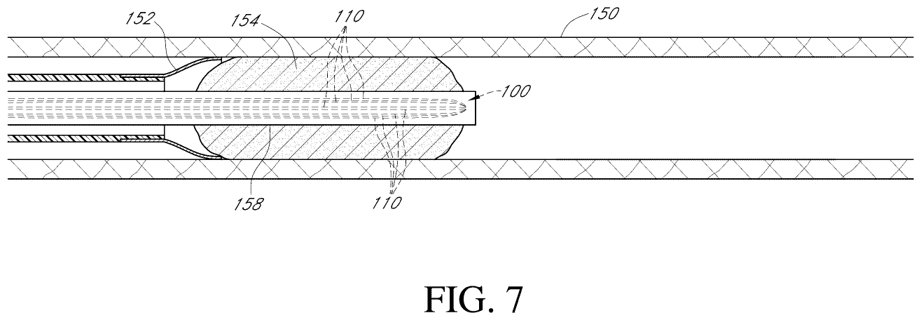

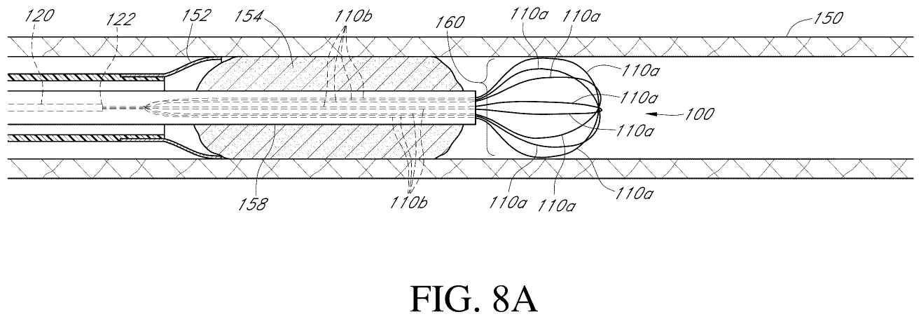

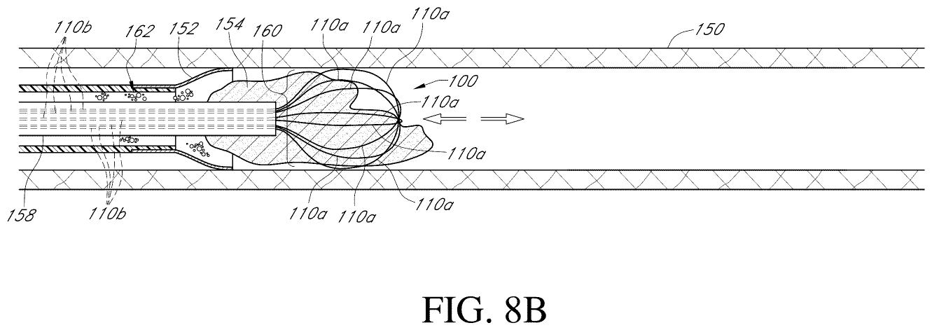

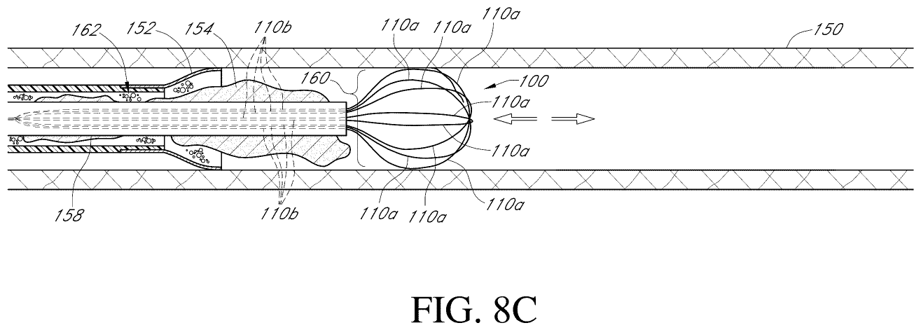

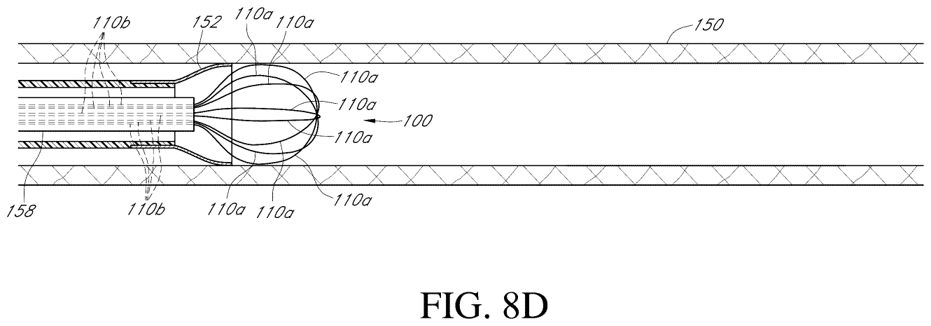

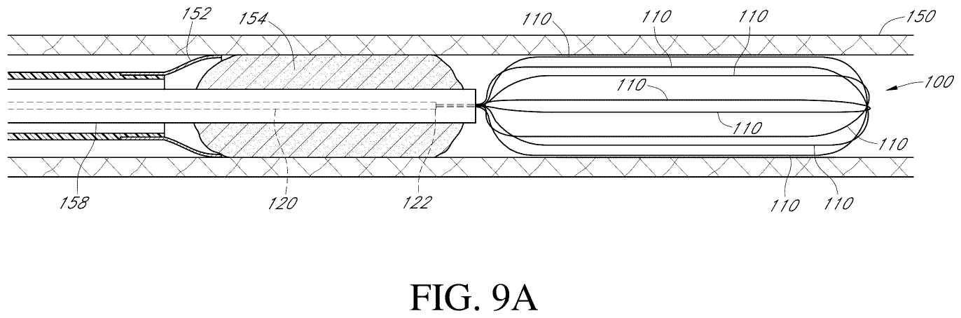

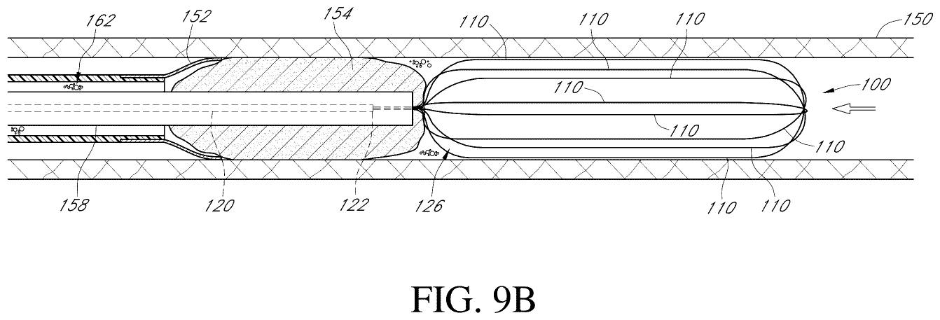

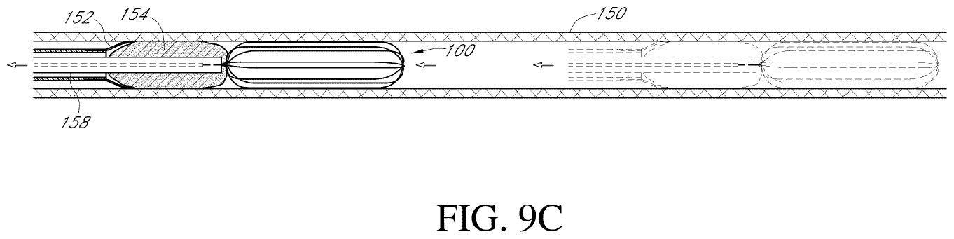

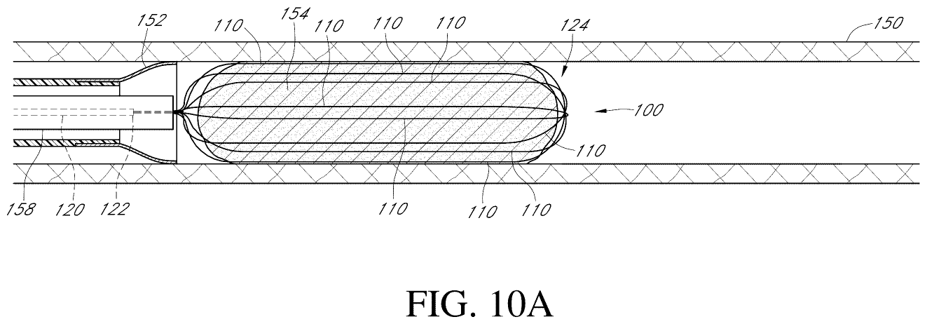

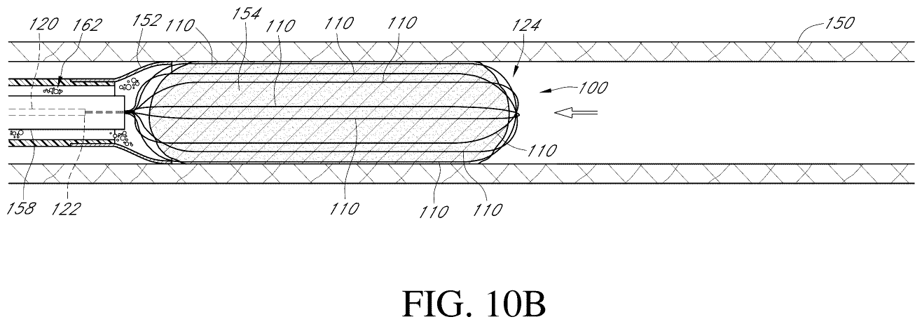

With the clot removal device 100 inside the microcatheter 158, the surgeon then advances the clot removal device 100 through the microcatheter 158 using the pusher wire 120 until the clot removal device 100 is at the location of the clot 154, as depicted in FIG. 7. It should be understood that the microcatheter 158 does not have to be exactly at the location depicted in FIG. 7 and, in fact, the microcatheter 158 may be inserted between the side of the clot 154 and the inner wall of the intracorporeal lumen 150. At this point, the surgeon can deploy the highly, versatile clot removal device 100 in any one of its possible configurations, some of which are, in this embodiment, 1) a partially constrained configuration (FIGS. 8A-D); 2) a clot trapping, or “en bloc”, configuration (FIGS. 9A-C) or 3) an enmeshed configuration (FIGS. 10A-B).

Referring now to FIGS. 8A-D, the surgeon has deployed the clot removal device 100 in a partially constrained configuration to engage in macerating the clot 154. Referring to FIG. 8A, just a portion of the clot removal device 100 has been advanced out of the end of the microcatheter 158 using the pusher wire 120. The portion of the clot removal device 100 that has been advanced out of the microcatheter 158 naturally expands. The surgeon, in this instance, continues to push the pusher wire 120 until the legs 110a of the clot removal device 100 contact the inner wall of the intracorporeal lumen 150. The legs 110 of the clot removal device 100, in this configuration, are partially constrained. A portion of the legs 110a have expanded in the intracorporeal lumen 150, while a portion of the legs 110b are still constrained within the microcatheter 158. In this configuration, the surgeon locks the clot removal device 100 into this partially constrained configuration by engaging the locking mechanism 142 in the hub assembly 140. The surgeon then uses the clot removal device 100 to macerate the clot 154. To do this, referring to FIGS. 8B-8C, the surgeon, using the pusher wire 120, pushes and pulls the partially constrained clot removal device 100 back (retracted proximally) and forth (advanced distally), engaging the clot 154. The clot removal device 100 engages the clot 154 with a cutting cage 160 formed by the ends of the legs 110a. The cutting edge 160, along with the activated aspiration catheter 152, act to macerate the clot 154 and break it up into clot fragments 162. The surgeon, in this example, by expanding the legs 110a of the clot removal device 100 to touch the inner wall of the intracorporeal lumen 150, ensures that the cutting cage 160 is cutting the maximum amount of the clot 154 it can with each pass. The surgeon continues to move the clot removal device 100 back (retracted proximally) and forth (advanced distally), continually macerating the clot 154, until the clot 154 is fully removed (FIG. 8D).

Referring now to FIGS. 9A-C, continuing the example from above, the surgeon is not having success macerating the clot 154 and decides he wants to try to trap the clot 154 instead and pull the clot 154 out. Because of the versatility of the clot removal device 100 of the present invention, the surgeon can do this on the spot without having to remove the clot removal device 100 from the patient to insert a new tool. Specifically, to do this, the surgeon unlocks the locking mechanism 142 and then pushes the pusher wire 120 distally to push more of the clot removal device 100 from the microcatheter 158 and further expand the clot removal device 100. In the example depicted in FIGS. 9A-C, the surgeon pushes the clot removal device 100 until the clot removal device 100 is fully expanded. Once in the clot trapping, or “en bloc”, configuration, the surgeon pulls the pusher wire 120 back towards him (retracts the pusher wire 120 proximally) until the cutting cage 126 engages the clot 154. With the clot removal device 100 engaging the clot 154, the clot 154 is now trapped between the clot removal device 100 and the aspiration catheter 152. At this point, the surgeon re-engages the locking mechanism 142 to lock the clot removal device 100 into this configuration. With the aspiration catheter 152 active, as depicted in FIG. 9C, the surgeon continues to pull the pusher wire 120 and aspiration catheter 152 back towards him (retracts the pusher wire 120 proximally) from the start position (shown in phantom) through the intracorporeal lumen 150 until the surgeon pulls the clot 154 out of the body 136. It should also be understood that the surgeon does not have to fully expand the clot removal device 100 to practice the trap technique. This technique can be used with a partially expanded clot removal device 100, as well. Further, it should also be understood that the surgeon does not have to try the partially constrained configuration first. The surgeon can go straight to the clot trapping, or “en bloc”, configuration at the outset.

Referring now to FIGS. 10A-B, continuing the example from above, the surgeon is not having success macerating the clot 154 or engaging it to pull it out “en bloc” and decides he wants to try to enmesh the clot 154 like a stent-triever instead and pull the clot 154 out that way. Again, because of the versatility of the clot removal device 100 of the present invention, the surgeon can do this on the spot without having to remove the clot removal device 100 from the patient to insert a new tool. Specifically, to do this, the surgeon, as before, unlocks the locking mechanism 142 and then pulls the pusher wire 120 proximally to re-sheath the clot removal device 100 in the microcatheter 158. With the clot removal device 100 re-sheathed in microcatheter 158 and the microcatheter 158 disposed within the clot 154, the surgeon pulls only the microcatheter 158 backwards (proximally) allowing the clot removal device 100 to expand within the clot 154, causing the clot removal device 100 to enmesh, and interdigitate with, the clot 154. Once in the enmesh configuration, the surgeon pulls the pusher wire 120 back towards him (retracts the pusher wire 120 proximally) until the proximal side of the clot removal device 100 engages the aspiration catheter 152. At this point, the surgeon re-engages the locking mechanism 142 to lock the clot removal device 100 into this configuration. With the clot removal device 100 enmeshed with the clot 154, the clot 154 is now stuck in the clot removal device 100. Similar to FIG. 9C while using the trap technique, with the aspiration catheter 152 active, the surgeon continues to pull the pusher wire 120 back towards him (retracts the pusher wire 120 proximally) from the start position through the intracorporeal lumen 150 until the surgeon pulls the clot 154 out of the body 136. During the extraction process, the distal cap 124 of the clot removal device 100 acts to prevent losing a grip on the clot 154 as it is being removed from the body 136. It should also be understood that the surgeon does not have to fully expand the clot removal device 100 to practice the enmesh technique. This technique can be used with a partially expanded clot removal device 100, as well. Further, it should also be understood that the surgeon does not have to try the partially constrained or the “en bloc” configurations first. The surgeon can go straight to the enmesh configuration at the outset.

Although certain embodiments and features of a clot and foreign body removal system and methods for use have been described herein, the scope of coverage of this patent is not limited thereto. On the contrary, this patent covers all embodiments of the teachings of the disclosure that fairly fall within the scope of permissible equivalents.

What is claimed is:

1. A system for retrieval of a clot or foreign body from within a vessel, the system comprising:
a retrieval assembly comprising an elongate wire and a distal member coupled to a distal end of the elongate wire, the distal member being moveable between at least a constrained configuration, a partially expanded configuration, and a fully expanded configuration;
an aspiration catheter comprising an elongate tubular body having proximal and distal ends and a lumen extending there between, the lumen having an inner diameter sized to receive the retrieval assembly within and further allow coaxial movement of the retrieval assembly relative to the tubular body when the distal member of the retrieval assembly is in at least one of the constrained, partially expanded, and fully expanded configurations;
wherein, in at least the partially expanded configuration, the distal member of the retrieval device is configured to engage a clot or foreign body within a vessel; and
wherein a distal tip of the distal end of the aspiration catheter is configured to make contact with at least a portion of the engaged clot or foreign body upon movement of the distal member towards the distal tip, the aspiration catheter being configured to communicate a vacuum along the length of the lumen from the proximal end to the distal tip to provide a suction force to the engaged clot or foreign body so as to capture the clot or foreign body for removal from within the vessel, thereby restoring blood flow through the vessel.

2. The system of claim 1, wherein the distal member of the retrieval assembly comprises a plurality of substantially elliptical wire elements coupled to the distal end of the elongate wire, each of the plurality of wire elements is configured to transition between the constrained, the partially expanded, and the fully expanded configurations.

3. The system of claim 2, wherein, in the constrained configuration, each of the wire elements is in a compressed state such that a length of each of the wire elements is substantially parallel with a longitudinal axis of the elongate wire.

4. The system of claim 3, wherein, when the distal member moves from the constrained configuration to at least one of the partially and fully expanded configurations, at least a portion of each wire element expands into a deployed state in a direction away from the elongate wire.

5. The system of claim 2, wherein, in at least one of the partially and fully expanded configurations, the plurality of wire elements forms a framework having a substantially spheroid shape.

6. The system of claim 2, wherein each of the plurality of wire elements comprises a shape memory alloy material.

7. The system of claim 6, wherein each of the plurality of wire elements comprises a medical grade nitinol alloy.

8. The system of claim 1, wherein, in the constrained configuration, the distal member has a first diameter and, in the fully expanded configuration, the distal member has a second diameter greater than the first diameter.

9. The system of claim 8, wherein the inner diameter of the lumen of the aspiration catheter body is greater than the second diameter of the distal member when in the fully expanded configuration.

10. The system of claim 1, wherein the distal member is detachable from the elongate wire of the retrieval assembly.

11. The system of claim 1, further comprising a microcatheter comprising an elongate tubular body having proximal and distal ends and a lumen extending there between, wherein the lumen has an inner diameter sized to receive the elongate wire and the distal member within when the distal member in the constrained configuration.

12. The system of claim 11, wherein the elongate wire and the microcatheter are coaxially moveable relative to one another.

13. The system of claim 12, wherein, upon movement of the elongate wire in a direction away from the proximal end and towards the distal end of the microcatheter body, at least a portion of the distal member is configured to be exposed from the microcatheter and further transition from the constrained configuration to at least the partially expanded configuration.

14. The system of claim 12, wherein, upon movement of the elongate wire in a direction away from the distal end and towards the proximal end and of the microcatheter body, at least a portion of the distal member is configured to be drawn within the inner lumen of the microcatheter body and further transition from at least the partially expanded configuration to the constrained configuration.

15. The system of claim 12, wherein, upon movement of distal end of the microcatheter in a direction away from the distal member and towards the elongate wire, at least a portion of the distal member is configured to be exposed from the microcatheter and further transition from the constrained configuration to at least the partially expanded configuration.

16. The system of claim 12, wherein, upon movement of distal end of the microcatheter in a direction away from the elongate wire and towards the distal member, at least a portion of the distal member is configured to be drawn into the inner lumen of the microcatheter body and further transition from at least the partially expanded configuration to the constrained configuration.

17. The system of claim 11, wherein the inner diameter of the aspiration catheter is sized to receive the microcatheter within and the microcatheter and aspiration catheter are coaxially moveable relative to one another.

18. The system of claim 11, wherein the microcatheter and the retrieval assembly are configured to be received within the inner lumen of the aspiration catheter body when the distal member is in at least one of the partial and fully expanded configurations and in engagement with the clot or foreign body.

19. The system of claim 1, wherein the aspiration catheter has a support assembly positioned along a length of the tubular body and in contact with a tubular wall of the tubular body, the support assembly configured to provide sufficient support and flexibility during catheter movement within the vessel and further provide support during removal of the clot or foreign material from within the vessel.

20. The system of claim 19, wherein the support assembly is configured to support the inner lumen of the aspiration catheter against collapse upon application of the suction force thereto.
